(19) United States
(12) Patent Application Publication
(10) Pub. No.: US 2005/0019269 A1
(43) Pub. Date: Jan. 27, 2005

(54) Mold eradication with Thyme solution and other essential oils

(75) Inventors: Lawrence M. Marks, Miami, FL (US); Victor A. Marcial-Vega, Miami, FL (US); Miguel A. Cisneros, Miami, FL (US)

Correspondence Address: ROBERT C. KAIN, JR., 750 SOUTHEAST THIRD AVENUE, SUITE 100, FT LAUDERDALE, FL, 333161153, US

(73) Assignee: Mold Eradication, LLC

(21) Appl. No.: 10/829,650

(22) Filed: Apr. 22, 2004

Related U.S. Application Data
(60) Provisional application No. 60/488,842, filed on Jul. 21, 2003.

(51) Int. Cl. A61L 9/04; A61K 35/78

(52) U.S. Cl. 424/45; 424/745; 424/747; 424/754; 424/770; 424/736; 424/742; 424/769; 424/778; 424/750

(57) ABSTRACT

The basic formulation to eradicate mold is a solution of 4 ounces of the essential oil of thyme mixed with one gallon of water. The mixture is sprayed, with a diffuser, into a room of a dwelling. The solution is misted over all surfaces. AC outlet vents are similarly sprayed. The AC inlet vent is also sprayed when the AC is turned ON. Preferably, the dwelling is tented and a small positive air pressure is maintained in the dwelling for 24 hours. The treatment is repeated with an enhanced solution of 4 oz. of essential oils mixed with one gallon of water. The mixture of essential oils is 50% by volume of thyme essential oil and the remaining 50% is equal parts of the essential oils of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert.

This is a regular patent application claiming the benefit of provisional patent application No. 60/488,842, filed Jul. 21, 2003.

The present invention relates to a mold eradication solution and process which acts as a fungicide to substantially eliminate mold from dwellings.

### OBJECT OF THE INVENTION

It is an object of the present invention to provide a mold eradication solution.

It is another object of the present invention to provide a system or a process for eradicating mold from a dwelling.

### SUMMARY OF THE INVENTION

The basic formulation is 4 ounces of the essential oil of thyme mixed with one gallon of water. The resulting mixture is sprayed, with a diffuser, into a room of a dwelling. The thyme solution is sprayed to mist over all surfaces. AC outlet vents are similarly sprayed for a predetermined period of time. The AC inlet vent is also sprayed when the AC is turned ON. Preferably, the dwelling is tented and a small positive air pressure is maintained in the dwelling for 24 hours. The dwelling treatment is repeated with an enhanced solution of 4 oz. of essential oils mixed with one gallon of water. The mixture of essential oils is 50% by volume of thyme essential oil and the remaining 50% is equal parts of the essential oils of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert.

### DETAILED DESCRIPTION OF INVENTION

Basic Formulation

The primary formulation is four (4) ounces of Thyme essential oil mixed in one gallon of spring water (herein the “thyme formula”), that is, about one part by volume of thyme essential oil and 32 parts of water. Tap water may be used. The oil does not break down or mix in the water and the resulting mixture should be dispersed, as discussed below, within 10 minutes of the initial mixing of the oil and water. An enhanced version of the formula is a mixture of 50% by volume of Thyme essential oil plus equal parts, by volume, constituting the remaining 50%, of essential plant oils of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert (herein the “enhanced thyme formula”). The four (4) oz. of the enhanced oil mixture if added to one (1) gallon of spring water.

All of the above essential aromatic plant oils are bought from different world sources. The essential oils have the following characteristics:
1. They are all found in nature
2. They grow in plants
3. Plants are processed in a way that removes their liquid, fat-soluble portions
4. These fat-soluble portions are chemically aromatic meaning they all contain a benzene ring that gives Thyme-Water mixture its unique properties
5. These properties include:
   a. Easy diffusion into the air and all aerated areas of a space
   b. A very pleasant smell
   c. Non-toxic
   d. Attachment to cell membranes of microorganisms such as mold (fungi), bacteria and their spores (such as anthrax spores) and viruses causing their immediate disruption and eradication (works on both viable and non-viable organisms)
   e. Proven benefit to humans by both decreasing the presence of disease causing microorganisms in the environment, stress reduction by smell recognition through brain pathways and increasing immune system function in humans by their anti-oxidant effect on the human body.
   f. Non-corrosive or staining of wood, paper, metal, rug fabric, tile and several other commonly found household materials therefore obviating the need to remove delicate furniture or structural materials that may be damaged by other effective but corrosive alternatives. This allows us to do minimal removal of affected structures.
      Chemical Composition of Components

The components of the formula are readily available with one North American company. Although here are many types of lavender, research and testing indicate that one particular kind of lavender from a particular company operates best.

Thyme White NF (Morocco) (Thymus spp.-Labiatae)

Obtained from the flowering tops, Thyme is distilled mainly in Spain and Israel although the herb is grown throughout Europe. It is a warm and stimulating oil that will relieve muscular pain. It is used to treat many kinds of infections in the skin, urinary tract and respiratory related. Because it is a strong antimicrobial it strengthens the immune response. Thymol is thyme's most active ingredient. Principal Constituents: 25-40% Thymol and carcacrol with borneol.

Cajeput (Malaysia) (Melaleuca leucadendrun-Myrtaceae)

This colorless oil is steam distilled from the leaves and buds of the Cajeput, which grows wild in the Far East. Main producers, Malaysia and Indonesia. The odor is strongly camphoraceous and medicinal. It is mainly used in inhalations, for respiratory infections including colds, coughs, sinus infections and sore throats. In vitro, it is a proven antimicrobial. Principal Constituents: Cineol (45% to 70%), aldehydes (benzoic, butyric, valeric, pinene, terpineol cineol, linalool, menthone, p-cymene, pinene, and triterpenic acid.

Cedarwood (U.S.A.)(Cedrus atlantica Manetti-Pinaceae)

This oil originated in Sri Lanka and is distilled from the leaf, its' color is yellow to light brown. Citronella is time tested and scientifically validated to be the best natural repellant for mosquitoes, ticks, fleas, and lice. Use in shampoos, natural insecticides and personal repellants. Principal Constituents: Terpenic hydrocarbons, cedrol and sesquiterpenes (cardinene).

Citronella (Ceylon)(Cymbopogon nardus-Gramineae).

Principal Constituents: Citronellol (20% to 40%), geraniol, citral, methyl-eugenol and various terpentine, bomeol and various terpenes.

Clove (bud) (Indonesia)(Eugenia caryophyllata-Myrtaceae)

This oil is colorless with a hint of yellow, it is obtained by distillation of the hand-picked and dried flower buds of the evergreen clove trees which are grown in the Far East and Africa. As it matures, the color turns to dark brown. It has a strongly stimulating affect and also has pain-relieving properties. It is antiseptic oil and is useful as a room fumigant. We are told that in the middle of the rainy season, the hot, humid atmosphere disperses the fragrance of the clove tree all over the land where they grow, and the lack of sickness in this areas is attributed to the medicinal scent emitted from this strong antiseptic tree. Principal Constituents: Phenols (70% to 0%), particularly eugenol. This was only isolated by Bonastre in 1827 and is one of the most antiseptics of the phenol family, two to four times stronger than any known. Acetyl eugenol (gives the fragrance), benzoic acid, benzyl benzoate, furfurol, sesquiterpenes (beta carophyllene), and vanillin.

Cypress (Spain)(Curpressus sempervirens-Cupressaceae)

This oil is distilled from the needles and twigs of the evergreen tree. The smell has freshness reminiscent of walking through a pine forest. It is colorless or very pale yellow, with a woodsy and balsamic, agreeable amber scent. Cypress oil is also produced in Kenya and Spain from Cupressus lusitanica and this too is used therapeutically. Cypress is an astringent oil, which tonifies the venous system and is useful for treating varicose veins, hemorrhoids, and broken capillaries. Useful in respiratory blends. Principal Constituents: Terpenes 65% (beta pinene and terpineol), cedrol, cypress camphor, some acids, and tannin.

Fir Needle (Asia)(Piceae aetheroleum-Fichtennadelol)

Obtained by distillation of the tips of the branches or twigs of the tree. It shares the same antiseptic, antiviral and antimicrobial properties as all of the pines distilled as Essential Oils. Excellent for respiratory synergies and/or blends. Use in aerotherapy and baths. Effective for rheumatic and neuralgic pains. Principal Constituents: Bornyl acetate, terpenes

Eucalyptus NF/NCC (Australia)(Eucalyptus globulus-Myrtaceaae)

Principal Constituents: Cineol or eucalyptol, from 70% to 80%; various aldehydes, ketones, sesquiterpenic alcohols, and terpenes, (approximately 250 different constituents in Eucalyptus). Used in a 2% spray solution, it kills 70% of the air borne Staphylococci bacteria. The essential oil of Eucalyptus has a much greater effect than Eucalyptol, its' main active principal constituent which is extracted and used in pharmaceuticals. It's effectiveness is primarily due to the action of two natural chemicals; aromadendrene and phellandrene when they come in contact with the oxygen in the air their chemical reaction produces ozone in which bacteria cannot live. Interaction with Other Drugs: “Eucalyptus Oil induces the enzyme system of the liver involved in the detoxification process. Therefore, the effects of other drugs can be weakened and/or shortened.” The Complete German Commission E. Monographs; Blumenthal, Busse, Goldberg, Gruenwald, Hall Klein, Riggins & Rister, copyright 1998, American Botanical Council, Austin, Tex.

Garlic (Hungary)(Allium sativum-Liliaceae)

Principal Constituents: Sulphur, iodine, silica, allicine and garlicine. Allistatines 1 & 2

Lavender NF/FCC (France)(Lavandula augustifolia/officinalis-Labiatae)

Principal Constituents: Alcohol's (borneol, geraniol, and linalool), esters, (geranyle and Linalyl), and terpenes (pinene and limonene). Lavender also contains a high proportion of phenol, which gives it very strong antiseptic and antibiotic properties.

Lemon NF/FCC (Italy)(Citrus limon-Rutaceae)

This oil is expressed from the peel of ripe lemons, grown mainly in Cyprus and California. It has a fresh truly reminiscent aroma of ripe lemons. Lemon Oil is highly antiseptic and has an astringent effect on the skin. It may be used by itself or in blends for; boils, broken capillaries, oily skin, herpes, and insect bites. Principal Constituents: Limonene, up to 90%, citral 3-5%, coumarines (bergamotine and libertine) and flavones (doismine and limotricine).

Lemongrass (India)(Cymbopogon citratus and flexuosus-Gramineae)

Principal Constituents: Citral (70% to 85%). C. flexuosus contains; citronellol, dipentene, farnesol, geraniol, linalool, limonene, methylheptenol, myrcene, n-deecylic aldehyde and nerol. C. citratus differs slightly, containing caprylics, citronellol, dipentene, farnesol, furfurol, geraniol, isopulegol, isovaleerianic aldehyde, 1-linalool, methylheptenone, myrcene, n-decyclic aldehyde, nerol, terpineol, and valeric esters.

Marjoram (French)(Origanum Marjorana)

Principal Constituents: Monoterpenes (40%); Sesquiterpenes; Monoterpenols (50%); Esters: terpenyle acetates, linalyle acetates. Properties: anti-bacterial, antiseptic, antispasmodic, arterial vasodilator, expectorant, sedative.

Niaouli (Spain)(Melaleuca viridiflora-Myrtaceae)

Distilled from the leaf, this oil appears very pale yellow to dark yellow and is very liquid. Niaouli is similar to Cajeput and Eucalyptus according to the aroma and therapeutic properties. Principal Constituents: Cineol 35-60%, eucalyptol, esters (butyric and isovaleerianic) limonene, pinene, and terpineol.

Onion (France)(Allii cepae bulbus-Liliaceae)

Principal Constituents: Aliin, sulfur compounds, peptides, and flavonoids.

Orange NF/FCC (U.S.A.)(Citrus aurantium sinenis-Rutaceeae)

Principal Constituents: 90% limonene with aldehydes, citral, citronellol, geraniol, linalool, methyl, anthranilate, nonyl alcohol and terpineol.

Oregano (Mediteranean)(Origanum Compactum & Vulgari)

Principal Constituents: Monoterpene(25%); Sesquiterpenes; Monoterpenols; Monoterpene phenols (60-70%): carvacrol, thymol; Methyl-ester phenols: methyl chavicol; Ketones. Properties: Anti-bacterial, anti-fungal, anti-parasitic, antiseptic to the respiratory system, anti-viral, and immune-stimulant.

Patchouli (Indonesia)(Pogossstemon cablin-Labiatae)

This oil is distilled mainly in Indonesia and the Philippines from the dried leaves of a small plant. It has a persistent, dry, woody smell. It is used especially for its fixative and fermenting properties in perfumery and relaxing blends. Patchouli is claimed to be an aphrodisiac and it may also be used in skin care blends to reduce scarring and to treat oily and problem skin. Principal Constituents: Patchoulol, 25% to 50%, sesquiterpenes (d-gauiene, norpatchoulenol, and patchoulene). Traces of benzoic and cinnamic aldehydes, cadinene, carvone, caryophyllene, coerulein, eugenol, humulene and seychellence.

Peppermint NF/NCC (U.S.A.)(Mentha piperita-Labiatae)

Distilled from the leaves and flowering tops that are native to Europe, it is known for its' decongestant, stimulating and refreshing properties. It is also used to treat many kinds of digestive upsets; nausea, flatulence, indigestion. Also good for migraines. Principal Constituents: Menthol 30% to 70%, terpenes (menthene, phellandrene, and limonene), 15% to 30% ketone (methone) and tannin.

Rosemary (France)(Rosmarinus officinalis-Labiatae)

Rosemary is found growing wild in the countries bordering the Mediterranean and has been used as a culinary and medicinal herb for centuries. The oil is refreshing, stimulating and is said to strengthen the memory, concentration and to relieve headaches. The oil ranges from colorless to slightly pale yellow-green, the aroma is similar to that of the crushed leaves; camphoric. It is also excellent for hair and scalp problems including hair loss and dandruff. Rosemary is a strong antioxidant, several studies have shown that it helps prevent the development of cancerous tumors in laboratory animals. Researchers at the University of Illinois in Urbana found that rosemary reduced the incidence of breast cancer in animals at high risk for developing the disease. “In the few studies done so far, rosemary has proven to be strong inhibitor of the development and growth of cancerous tumors,” says Dr. Chi-Tang Ho, Ph.D., Professor, Department of Food Science at Rutgers University in New Brunswick, N.J.. Principal Constituents: Bomeol 15%, camphene, camphor, cineol, lineol, pinene, resins and saponin.

Rosewood (Brazil)(Aniba rosaeodora)

Native to the Amazon region, this oil is extracted by distillation from the wood chips, pale yellow in color with a flower/wood smell. Known for its' immune boosting and relaxing qualities, this essential oil is a good addition to any blend. As a mild analgesic, it is effective in headache synergies. Rosewood is an antiseptic and antibacterial useful in aerotherapy. It is reputed as a cell regenerator, which makes it valuable in skin care synergies and/or blends. Principal Constituents: Linalol (80% to 90%), geraniol, and terpineol.

Tea Tree/Ti-Tree (Australia)(Melaleuca altemifolia-Myrtaceae)

Principal Constituents: Terpenes, 50-60%, cineol, sesquiterpenes, and sesquiterpenic alcohols.

Ylang-Ylang 1^{st} grade extra (Indonesia) (Cananga odorata-Anonaceae)

This oil is distilled from the fresh flowers of the trees grown mainly on the Island of Comores, off the coast of East Africa and Indonesia. It has a powerful and exotic, floral smell and has a relaxing affect on the nervous system. It is traditionally used as an aphrodisiac and for the treatment of high blood pressure. Principal Constituents: Alpha pinene, benzoic acid, cadinene, caryophyllene, cresol, eugenol, isoeugenol, 5-7% linalyl acetate, 8-10% lynalylbenzoate and 30-32% linalool and geraniol.

Other essential oil: Vetivert.

Mold Eradication Protocol

The mold eradication protocol has three main components: 1. Initial evaluation of a structure; 2. Treatment planning and treatment; and 3. Follow-up evaluation for efficacy. “Mold” is defined as a common name for fungi that grow in a filamentous fashion and reproduce by means of spores; all molds are fungi, but not all fungi are considered “molds.”

Initial Evaluation

Mold causes problems in several ways. It can either cause physical symptoms in human beings and other animals or it can destroy property by growing on carpets, drywall, wood, wallpaper, furniture or other surfaces. Mold is present in ambient air as part of its natural habitat but when beneficial conditions exist, mainly moisture and water, it will overgrow. The most common reason for this overgrowth is the presence of a water leak inside of a home from either poor perimeter insulation or leaky inside plumbing pipes. For these reasons, the first step in the assessment of a home suspected of containing overgrowth of mold is identification of the cause of a water leak which will promote continuing growth.

The second step is evaluation of the damage done to the dwelling by either mold, water or both.

The third step is to document the presence of mold both quantitatively and qualitatively.

There are two ways of documenting the presence of mold: qualitatively and quantitatively.

Qualitative Tests

Qualitative testing tells identifies what mold may be present on a particular surface but it does not indicate how much mold is present. One must assess the quantity of mold present to both make a comparison with the outside air (this is called a “control”) and determine if the quantities of mold found inside the dwelling are within the normal parameters or not and to evaluate the effectiveness of the treatment. Qualitatively tests are:
A. Swab test—A swab is taken of the surface in question to confirm the presence of viable mold.
B. Tape test—Tape is pressed lightly against surfaces such as walls, carpet or floor. This is then read on a microscope for the presence of viable and non-viable mold.

It should be noted that “viable mold” is mold that is capable of reproducing and “non-viable mold” is mold that cannot reproduce.

Quantitative Tests

Quantitative tests are the main mode of testing used to determine the presence of mold and evaluate the effectiveness of the treatment.
1. Air-O-Cell—This measures the amount of viable and non-viable mold in the air.
2. Andersen Impactor—measures the amount of viable mold in the air

Air-O-Cell and Andersen Impactor are commercially available products. Both of the above noted quantitative tests are done in each room of the building that is suspected of having mold, fungus growth or contamination. A control sample of the outside air is always obtained for comparison.

Treatment Criteria

Many times, persons experiencing allergenic symptoms related to mold growth are frequently the first indication that treatment of the environment and/or physical structure needs to be accomplished. However, these symptoms must be accompanied by objective criteria that will both confirm the presence of mold and determine the effectiveness of the treatment after implementation.

A fungal concentration of mold (viable, non-viable or both) greater than a third (33%) of the mold concentration of the outside ambient air while the air conditioner is in the building or structure is ON and at temperatures of 70-72 degrees Fahrenheit is considered abnormal for both viable or non-viable mold. When these abnormalities are present, mold eradication treatment is indicated.

Treatment

1. Repair of water leak(s)

2 Removal of structure(s) damaged by water or mold

3. Tenting of the dwelling

4. Applying 2-3 pounds of positive pressure above the atmosphere in the building or structure

5. Spraying with Thyme Only Formula

Composition: 4 ounces of Thyme essential oil mixed with one gallon of spring water.

The Thyme Only composition is used in an electrical diffuser capable of producing a 10 feet forward directed or long and one foot wide mist in an enclosed space with particles ranging in size from 1-15 angstroms wide. All surfaces are sprayed and misted (mist covered) with this mixture.

The structure's air conditioner is then turned OFF and all AC outlet grills are removed. A three (3) foot extension wand is introduced each AC outlet and the proximal portion of the duct is treated with the Thyme only composition as sprayed by the diffuser for one (1) minute. The process is repeated for each AC duct outlet such that all vents are misted. The building's air conditioner is then turned ON. The air conditioner intake is then sprayed with the Thyme only solution for 5-10 minutes at a rate of 2 cc per minute.

Personnel may vacate the premises and the building is tent sealed with 2-3 pounds of positive air pressure. Twenty-four (24) hours after this intensive spraying, mold cultures and samples are gathered and mold testing is repeated.

The above cycle is repeated with a mixture the Thyme Enhanced Composition of thyme, oregano, lavender, ti-tree, onion, garlic, cedarwood, orange, eucalyptus and lemongrass essential oils until all mold is within normal levels (less than a third of outside air).

Maintenance

Optionally, the occupant may continue the mold eradication treatment with a maintenance schedule. After initial mold eradication treatment and documentation of mold eradication, a maintenance program with a daily application is suggested to prevent further growth of mold or other organisms.

A 24 hour diffuser is installed at the intake of the air conditioning system and filled with a mixture of thyme, eucalyptus, garlic, onion, ti- tree, orange, cedarwood, lavender, lemongrass and oregano. Every five (5) minutes, a 30 second pulse of spray is released into the air at the intake of the AC system.

The above processes may be repeated as often as necessary until all mold is eradicated as indicated by the quantitative testing.

Experimental Testing

In excess of 50 tests were performed in the following project to show the effectiveness of the formula.

An apartment was chosen on the eighteenth floor of an apartment complex in South Florida. This apartment had water leaks that had caused infestation of mold in the walls of several of the rooms. Air samples were taken of the following areas: 1. Outside air (control); 2. Downstairs bedroom(first floor); and 3. Upstairs bedroom(second floor). These three areas were tested at different intervals and different dates for both viable and non-viable mold counts (Air-O-Cell system) testing and viable-only testing (Andersen Impactor) using petri dishes.

Viable mold spores are spores that can reproduce when conditions are appropriate. Non-viable mold spores cannot reproduce but may still be alive. Both are capable of causing structural damage and disease in humans and other animals. Viable mold has the potential to cause more damage than non-viable mold since it has the ability to reproduce.

The initial baseline results showed the following:
1. Air-O-Cell (Results show both viable and non-viable mold spores)
2. Outside air—183 counts per cubic meter (Counts/cubic meter)
3. Downstairs bedroom—731 (Counts/cubic meter)
4. Upstairs Bedroom—2,377 (Counts/cubic meter)

This data indicates that the upstairs bedroom's air was more contaminated than the downstairs bedroom's. It also indicates the indoor mold is higher than the outside levels.

Andersen Impactor (Results show viable mold spores only):
1. Outside Air—43 Colony Forming Units per cubic meter(CFU/cubic meter)
2. Downstairs Bedroom—136 CFU/cubic meter
3. Upstairs Bedroom—93 CFU/cubic meter

Over the following 3 days, the contaminated rugs, walls and partitions were removed. Air samples were repeated thereafter and showed: (a) Air-O-Cell (viable and non-viable): Both bedrooms samples were so high, they were overloaded and could not be counted; (b) Andersen Impactor: Both rooms showed levels above 2,143 CFU/cubic meter.

After the above base-line samples were obtained, the entire dwelling was sprayed in the manner described above. Three (3) days after a single application of the Thyme Only formula, per the diffusion spray and AC vent cleaning, quantitative and qualitative mold tests were again conducted. The following results were obtained:

Air-O -Cell: Both lower and upper bedroom showed a marked decrease to 183 Counts/cubic meter. The outside ground level control was 366 counts/cubic meter.

Andersen Impactor: (a) Lower bedroom—64 cfu/cubic meter; (b) Upper bedroom—>88 cfu/cubic meter.

All of the above readings were considerably smaller than the levels obtained the day of the treatment. Conditions were purposely unfavorable. The building's air conditioners were always OFF and temperatures were always between 85-95 degrees Fahrenheit with relative atmospheric humidity of 75-82%. These conditions of no AC, high temperatures and high humidity promote mold growth. In addition, there was not a tight seal around the apartment unit since there were connections with adjoining apartments and doors were not sealed. This type of air leakage tends to dilute the effectiveness of the Thyme formula.

Better results can be obtained with: (a) constant use of the air conditioner at 70 degrees Fahrenheit; and (b) tight seal or tenting of the household.

These tests show that one single treatment with the Thyme Only formula under the worse known adverse conditions such as extremes of high relative humidity and high temperature and lack of an adequate seal in the building will dramatically reduce the levels of mold inside the environment to levels equal or less to outside air levels.

The dwelling is certified as free of mold when the quantitative tests reveal that the inside air contains a third or less of the outside air's level of mold. The unit is then ready for any structural remediation needed. Because the formula eliminates viable and non-viable mold, the mold eradication system requires less removal of physical structures affected than conventional methods that sometimes requires complete gutting of the dwelling. The system only requires removal of destroyed portions of the dwelling and carpets.

In addition to the foregoing, the following processes should eradicate mold:

1. Cold thermal diffusion (man operated); a mixture of selected Essential Oils, suspended in water. Then an atomizer or spray bottle is used to spray the air of a room or an entire house once or twice a day as needed to keep the air full of microorganisms.

2. Timed release diffusion of the water and Thyme essential oil mixture (enhanced) in AC room; a timer is connected to a drum or fan device filled with a mixture of the selected Essential Oils suspended in water. Timer is set for desire sequence diffusion, example; every 15 or 30 minutes, spraying for 5-10 seconds. AC system will carry mixture throughout area. This is done as a maintenance daily treatment of the entire house air.

3. Rock salt saturated diffusion; a container full of rock salt is saturated with desired Essential Oil mix, the salt rocks will absorb the Essential Oils. The rocks are then placed in an open container in selected areas inside or outside the house for maintenance treatment of the air on a daily basis.

The claims appended hereto are meant to cover modifications and changes within the scope and spirit of the present invention.

1. A solution for eradication of mold comprising about one part by volume of thyme essential oil and 32 parts of water.

2. A solution for the eradication of mold comprising about one part by volume of thyme essential oil and 64 parts of water and one part by volume of substantially equal amounts of the essential oil of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert.

3. A process for eradication of mold in a dwelling comprising:
dispersing an aerosol of a solution of about one part by volume of thyme essential oil and 32 parts of water in said dwelling.

4. A process as claimed in claim 1 including substantially sealing said dwelling after dispersal of said solution and establishing a positive pressure greater than the ambient atmospheric pressure in said dwelling for a predetermined period of time.

5. A process as claimed in claim 3 including misting substantially all surfaces in said dwelling with said solution.

6. A process as claimed in claim 3 wherein said solution is a first solution, and including, after the dispersal of said first solution, dispersing, in said dwelling, an aerosol of a second solution of about one part by volume of thyme essential oil and 64 parts of water and one part by volume of substantially equal amounts of the essential oil of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert.

7. A process as claimed in claim 3 wherein said dwelling includes heat, ventilation and air conditioning ducts throughout said dwelling, and the process includes the aerosol dispersion of said solution in ducts in said dwelling for a predetermined period of time.

8. A process as claimed in claim 7 including misting substantially all surfaces in said dwelling with said solution.

9. A process as claimed in claim 8 including substantially sealing said dwelling after dispersal of said solution and establishing a positive pressure greater than the ambient atmospheric pressure in said dwelling for a predetermined period of time.

10. A process as claimed in claim 9 wherein said solution is a first solution, and including, after the dispersal of said first solution and after a predetermined period of time, dispersing, in said dwelling, an aerosol of a second solution of about one part by volume of thyme essential oil and 64 parts of water and one part by volume of substantially equal amounts of the essential oil of: cajeput, cedarwood, citronella, clove, cypress, fir-needle, eucalyptus, garlic, lavender, lemon, lemongrass, marjoram, niaouli, onion, orange, oregano, patchouli, peppermint, rosemary, rosewood, tea tree, y-lang and vetivert.
